(19) United States
(12) Patent Application Publication
(10) Pub. No.: US 2014/0163673 A1
(43) Pub. Date: Jun. 12, 2014

(54) PROSTHETIC HEART VALVE LEAFLET ADAPTED FOR EXTERNAL IMAGING

(71) Applicant: W. L. Gore & Associates, Inc., Newark, DE (US)

(72) Inventors: William C. Bruchman, Camp Verde, AZ (US); Cody L. Hartman, Flagstaff, AZ (US)

(73) Assignee: W. L. Gore & Associates, Inc., Newark, DE (US)

(21) Appl. No.: 14/182,044

(22) Filed: Feb. 17, 2014

Related U.S. Application Data
(63) Continuation-in-part of application No. 13/485,823, filed on May 31, 2012.
Application No. 13/485,823 is a continuation-in-part of application No. 13/078,774, filed on Apr. 1, 2011.
Application No. 13/078,774 is a continuation-in-part of application No. 13/078,774, filed on Apr. 1, 2011.
(60) Provisional application No. 61/800,402, filed on Mar. 15, 2013.
(60) Provisional application No. 61/887,089, filed on Oct. 4, 2013.

(51) Int. Cl. A61L 27/50; A61L 27/48; A61L 27/56; A61F 2/24

(52) U.S. Cl. CPC A61L 27/50; A61F 2/24; A61L 27/48; A61L 27/56
USPC 623/2.42

(57) ABSTRACT

Embodiments provided herein are related to prosthetic heart valve leaflets comprising one or more imageable elements that allow for visualization of the movement of the leaflets using imaging techniques, such as, but not limited to fluoroscopy, x-ray, ultrasound, and MRI. When visualized using visualization techniques, the movement of the imageable element is directly related to the movement of the leaflet to which it is coupled, and therefore the movement of the leaflet may be determined.

### FIELD

The present disclosure relates generally to prosthetic heart valves having prosthetic heart valve leaflets, and more specifically, prosthetic heart valve leaflets that are adapted for external imaging.

### BACKGROUND

Prosthetic heart valves have been developed that attempt to mimic the function and performance of a native valve. Flexible leaflets are fabricated from biological tissue or synthetic materials. The function of the valve needs to be accessed after surgical or transcatheter placement in order to determine the effectiveness of the procedure.

A number of techniques have been used in an attempt to assess the function of the leaflets of the prosthetic valve after implantation.

Two-dimensional transthoracic or transesophageal echocardiography is a technique that uses ultrasound to display a cross sectional “slice” of the beating heart, including the valve and valve leaflets. Three-dimensional echocardiography is being studied to provide a stereoscopic image. Prosthetic heart valve leaflets have a very low mass and have a relatively complex structure and therefore are not easily visualized via echocardiography. Accurate interpretation of the image requires a high skill level on the part of the echocardiographer. A more objective and quantitative evaluation of leaflet function that would reduce the subjectivity in the interpretation of images is needed.

Fluoroscopy, an x-ray imaging technique, is routinely used during percutaneous interventions in the catheter laboratory. In particular, fluoroscopy is used to guide and place transcatheter heart valves. Radiopaque markers that are visible under fluoroscopy may be coupled to the delivery catheter and the prosthetic valve frame to help in placement of the prosthetic valve. Biological tissues are not easily visualized directly by fluoroscopy. Contrast agents are commonly injected into the blood stream to visualize the flow path through the heart valve. Visualization of the blood stream is used to indirectly determine the function of the leaflets of the prosthetic heart valve.

Prosthetic valve leaflets made from synthetic materials, such as polymers, including polyurethane and fluoropolymer, among others, are also poorly visualized using fluoroscopic techniques.

There exists a need in the art to provide a visual confirmation of proper prosthetic heart valve leaflet function using external imaging techniques, particularly after transcatheter placement that allows for a more objective and quantitative evaluation of leaflet function that would reduce the subjectivity in the interpretation of the images.

### BRIEF DESCRIPTION OF THE DRAWINGS

Exemplary embodiments will be described in conjunction with the accompanying drawing figures in which like numerals denote like elements and:

FIG. 1A is a top view of an embodiment of a valve in a closed configuration, in accordance with an embodiment;

FIG. 1B is an axial view of the embodiment of the valve of FIG. 1A in an open configuration, in accordance with an embodiment;

FIG. 2 is a perspective view of the embodiment of the valve of FIG. 1A in a closed configuration, in accordance with an embodiment;

FIG. 3 is a perspective view of an embodiment of a valve frame, in accordance with an embodiment;

FIG. 4A is a perspective view of a valve in a closed configuration having a leaflet with a radiopaque marker, in accordance with an embodiment;

FIG. 4B is an axial view of a valve in a closed configuration having a leaflet with a plurality of radiopaque markers, in accordance with an embodiment;

FIG. 4C is an axial view of a valve in a closed configuration having a leaflet with a radiopaque marker, in accordance with an embodiment;

FIG. 4D is an axial view of a valve in a closed configuration having a leaflet comprising radiopaque material, in accordance with an embodiment;

FIG. 5 is a side view of a leaflet frame coupled to a mandrel in the process of having a film wound thereon defining layers, with a radiopaque marker contained between at least two of the layers of film, in accordance with an embodiment; and

FIG. 6 is a cross-sectional view of a radiopaque marker between layers of film, in accordance with an embodiment.

### DETAILED DESCRIPTION

Persons skilled in the art will readily appreciate that various aspects of the present invention may be realized by any number of methods and apparatus configured to perform the intended functions. Stated differently, other methods and apparatus may be incorporated herein to perform the intended functions. It should also be noted that the accompanying drawing figures referred to herein are not all drawn to scale, but may be exaggerated to illustrate various aspects of the present invention, and in that regard, the drawing figures should not be construed as limiting.

Although the embodiments herein may be described in connection with various principles and beliefs, the described embodiments should not be bound by theory. For example, embodiments are described herein in connection with prosthetic valves, more specifically cardiac prosthetic valves. However, embodiments within the scope of this disclosure can be applied toward any valve or mechanism of similar structure and/or function. Furthermore, embodiments within the scope of this disclosure can be applied in non-cardiac applications.

The term leaflet as used herein in the context of prosthetic valves is a component of a one-way valve wherein the leaflet is operable to move between an open and closed position under the influence of a pressure differential. In an open position, the leaflet allows blood to flow through the valve. In a closed position, the leaflet substantially blocks retrograde flow through the valve. In embodiments comprising multiple leaflets, each leaflet cooperates with at least one neighboring leaflet to block the retrograde flow of blood. The pressure differential in the blood is caused, for example, by the contraction of a ventricle or atrium of the heart, such pressure differential typically resulting from a fluid pressure building up on one side of the leaflets when closed. As the pressure on an inflow side of the valve rises above the pressure on the outflow side of the valve, the leaflets opens and blood flows therethrough. As blood flows through the valve into a neighboring chamber or blood vessel, the pressure on the inflow side equalizes with the pressure on the outflow side. As the pressure on the outflow side of the valve rises above the blood pressure on the inflow side of the valve, the leaflet returns to the closed position generally preventing retrograde flow of blood through the valve.

The term membrane as used herein refers to a sheet of material comprising a single composition, such as, but not limited to, expanded fluoropolymer and synthetic polymer having a structure defining fibers, such as, but not limited to, porous polyethylene.

The term composite material as used herein refers to a combination of a membrane, such as, but not limited to, expanded fluoropolymer, and an elastomer, such as, but not limited to, a fluoroelastomer. The elastomer may be imbibed within a porous structure of the membrane, coated on one or both sides of the membrane, or a combination of coated on and imbibed within the membrane.

The term laminate as used herein refers to multiple layers of membrane, composite material, or other materials, such as elastomer, and combinations thereof.

The term film as used herein generically refers to one or more of the membrane, composite material, or laminate.

The term leaflet window is defined as that space that a frame defines from which a leaflet extends. The leaflet may extend from frame elements or adjacent to frame elements and spaced apart therefrom.

The terms native valve orifice and tissue orifice refer to an anatomical structure into which a prosthetic valve may be placed. Such anatomical structure includes, but is not limited to, a location wherein a cardiac valve may or may not have been surgically removed. It is understood that other anatomical structures that may receive a prosthetic valve include, but are not limited to, veins, arteries, ducts and shunts. Although reference is made herein to replacing a native valve with a prosthetic valve, it is understood and appreciated that a valve orifice or implant site may also refer to a location in a synthetic or biological conduit that may receive a valve for a particular purpose, and therefore the scope of the embodiments provided herein is not limited to valve replacement.

As used herein, “couple” means to join, connect, attach, adhere, affix, or bond, whether directly or indirectly, and whether permanently or temporarily.

Embodiments herein include various apparatus, systems, and methods for a prosthetic valve suitable for surgical and transcatheter placement, such as, but not limited to, cardiac valve replacement. The valve is operable as a one-way valve wherein the valve defines a valve orifice into which leaflets open to permit flow and close so as to occlude the valve orifice and prevent flow in response to differential fluid pressure.

Embodiments herein include various apparatus, systems, and methods for a prosthetic valve suitable for, such as, but not limited to, cardiac valve replacement. The valve is operable as a one-way valve wherein the valve defines a valve orifice into which leaflets open to permit flow and close so as to occlude the valve orifice and prevent retrograde flow.

Embodiments provided herein are related to prosthetic heart valve leaflets comprising one or more imageable elements that allow for visualization of the movement of the leaflets using imaging techniques, such as, but not limited to fluoroscopy, x-ray, ultrasound, and MRI. When visualized using visualization techniques, the movement of the imageable element is directly related to the movement of the leaflet to which it is coupled, and therefore the movement of the leaflet may be determined.

Embodiments provided herein are related to prosthetic heart valve leaflets comprising one or more radiopaque markers. The radiopaque marker is operable to be visualized using x-ray techniques, such as, but not limited to fluoroscopy or other radiographic technique. When visualized using x-ray techniques, the movement of the radiopaque marker is directly related to the movement of the leaflet to which it is coupled, and therefore the movement of the leaflet may be determined.

Embodiments provided herein are related to prosthetic heart valve leaflets comprising one or more echogenic elements. The echogenic element is ultrasonically reflective, that is, operable to be visualized using ultrasound imaging techniques. When visualized using ultrasound imaging techniques, the movement of the echogenic element is directly related to the movement of the leaflet to which it is coupled, and therefore the movement of the leaflet may be determined.

In accordance with embodiments presented herein, a prosthetic valve comprises a plurality of polymer leaflets. The polymer leaflets comprise a laminate of multiple layers of membrane, composite material, or other materials, such as elastomer, and combinations thereof. One or more imageable elements are coupled to and contained within the laminate lying between two of the multiple layers of membrane or composite material. The imageable element is operable to provide minimal structural influence of the leaflet so as to not impede leaflet movement. Since the imageable element is fully contained within the laminate layers, the imageable element remains permanently coupled to the leaflet. Further, since the imageable element is fully contained within the laminate layers, the imageable element is not exposed to the blood stream.

### Valve

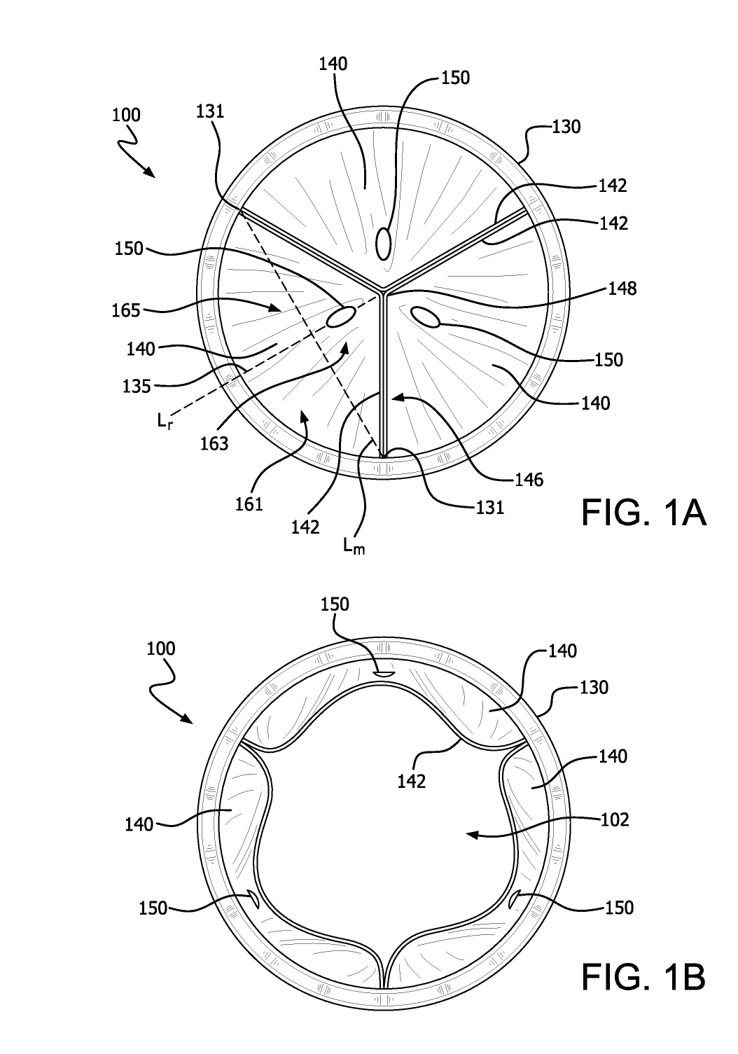

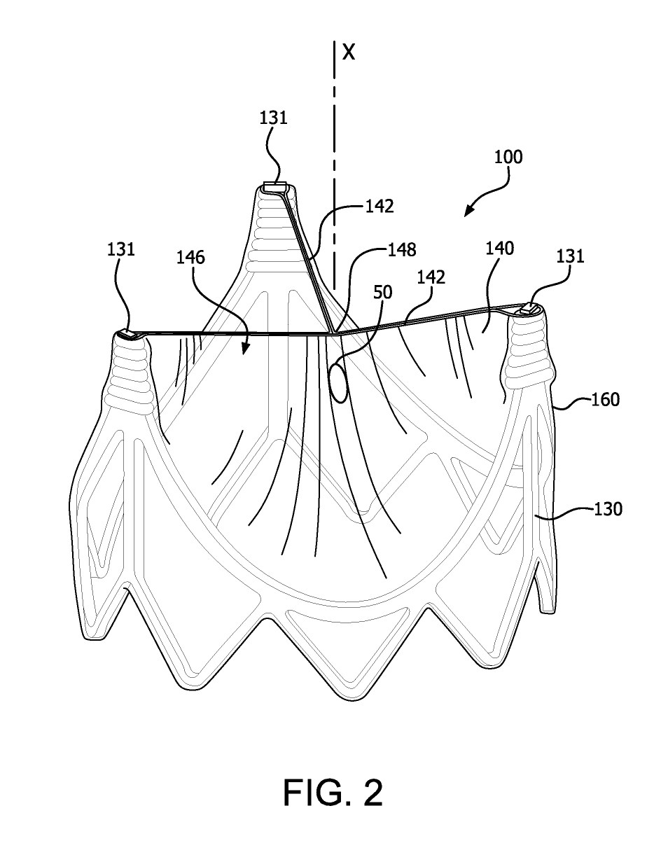

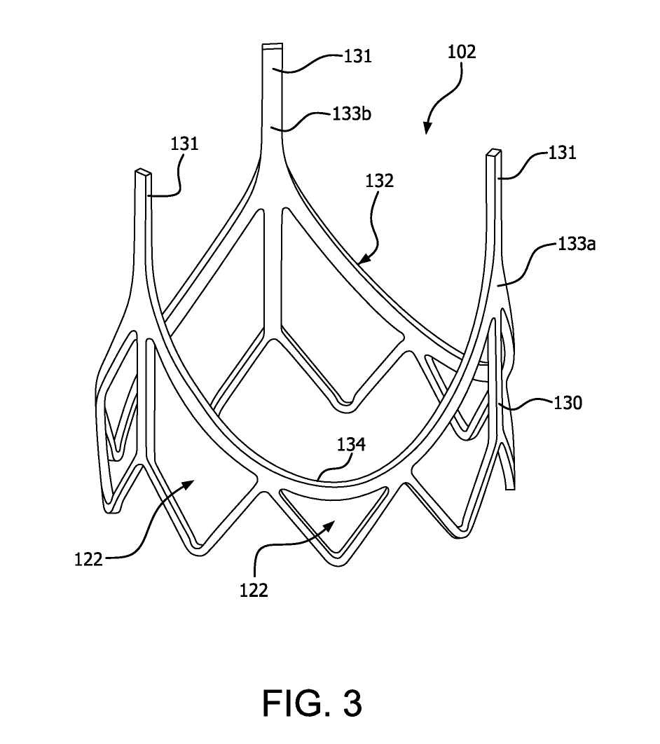

FIGS. 1A and 1B are axial views of a valve 100 in the closed and open condition, respectively, in accordance with an embodiment. FIG. 2 is a perspective view of the valve 100 in the closed condition. The valve 100 comprises a frame 130 and a film 160 covering the frame 130 forming the leaflets 140 coupled to the frame 130, in accordance with an embodiment. The leaflets 140 comprise an imageable element 150. FIG. 3 is a perspective view of the frame 130, in accordance with an embodiment. The size, shape and configuration of the valve 100 presented herein is by way of example.

### Frame

FIG. 3 is a perspective view of the frame 130 in the embodiment of FIGS. 1A and 1 B. The frame 130 is a generally tubular member defining a valve orifice 102 and providing structural, load-bearing support to the leaflet 140. In addition, the frame 130 can be configured to provide positive engagement to the recipient tissue at the implantation site.

The frame 130 can comprise any metallic or polymeric biocompatible material. For example, the frame 130 can comprise a material, such as, but not limited to nitinol, cobalt-nickel alloy, stainless steel, and polypropylene, acetyl homopolymer, acetyl copolymer, ePTFE, other alloys or polymers, or any other biocompatible material having adequate physical and mechanical properties to function as described herein.

By way of example, and as illustrated in the embodiments of FIGS. 1A-B, 2 and 3, the frame 130 defines a stent having apertures 122. The open framework of the stent can define any number of features, repeatable or otherwise, such as geometric shapes and/or linear or meandering series of sinusoids. An open framework can be etched, cut, laser cut, or stamped into a tube or a sheet of material, with the sheet then formed into a substantially cylindrical structure. In other embodiments, the frame 130 can have a solid wall. Alternatively, an elongated material, such as a wire, bendable strip, or a series thereof, can be bent or braided and formed into a substantially cylindrical structure. For example, the frame 130 can comprise a stent or stent graft type structure or a conventional sewing frame as found in bioprosthetic valves known in the art.

In accordance with embodiments, the frame 130 can be configured to provide positive engagement to an implant site. In another embodiment, the valve 100 further includes a sewing cuff (not shown) coupled about the frame 130, that is operable to accept suture so as to be sewn to a tissue orifice as is known in the art. It is understood that conventional surgical and transcatheter techniques to implant prosthetic valves can be used to implant the valve 100.

The frame 130 comprises three interconnected U-shaped portions 132. Each of the U-shaped portions 132 defines a base 134. The U-shaped portions 132 intersect with an adjacent U-shaped portion defining a post 131. The frame 130 as shown in FIG. 3 comprises three U-shaped portions 132 and three posts 131, upon each of which a leaflet 140 is coupled as shown in FIG. 2.

The frame 130 can comprise, such as, but not limited to, an elastically deformable metallic or polymeric biocompatible material. The frame 130 can comprise a shape-memory material, such as nitinol, a nickel-titanium alloy. Other materials suitable for the frame 130 include, but not limited to, other titanium alloys, stainless steel, cobalt-nickel alloy, polypropylene, acetyl homopolymer, acetyl copolymer, other alloys or polymers, or any other biocompatible material having adequate physical and mechanical properties to function as a frame 130 as described herein.

As will be described in more detail below, a film 160 is disposed over each of the three leaflet windows 137 to form a leaflet 140.

### Film

A film 160 generically refers to one or more of the membrane, composite material, or laminate as previously defined. The leaflets 140 are comprised of the film 160. Details of various types of film 160 are discussed below. In an embodiment, the film 160 can be formed from a generally tubular material to couple the frame 130 and to form the leaflets 140. As will be discussed below, the laminate comprises a number of layers of membrane and/or composite material, with the imageable element 150 being coupled and contained within at least two layers of membrane and/or composite material.

In an embodiment, the film 160 comprises a biocompatible polymer that is combined with an elastomer, referred to as a composite. A material according to one embodiment includes a composite material comprising an expanded fluoropolymer membrane, which comprises a plurality of spaces within a matrix of fibrils, and an elastomeric material. It should be appreciated that multiple types of fluoropolymer membranes and multiple types of elastomeric materials can be combined to form a laminate while remaining within the scope of the present disclosure. It should also be appreciated that the elastomeric material can include multiple elastomers, multiple types of non-elastomeric components, such as inorganic fillers, therapeutic agents, radiopaque materials, and the like while remaining within the scope of the present disclosure.

In accordance with an embodiment, the composite material includes an expanded fluoropolymer material made from porous ePTFE membrane, for instance as generally described in U.S. Pat. No. 7,306,729 to Bacino.

The expandable fluoropolymer, used to form the expanded fluoropolymer material described, may comprise PTFE homopolymer. In alternative embodiments, blends of PTFE, expandable modified PTFE and/or expanded copolymers of PTFE may be used. Non-limiting examples of suitable fluoropolymer materials are described in, for example, U.S. Pat. No. 5,708,044, to Branca, U.S. Pat. No. 6,541,589, to Baillie, U.S. Pat. No. 7,531,611, to Sabol et al., U.S. patent application Ser. No. 11/906,877, to Ford, and U.S. patent application Ser. No. 12/410,050, to Xu et al.

The expanded fluoropolymer membrane can comprise any suitable microstructure for achieving the desired leaflet performance. In accordance with an embodiment, the expanded fluoropolymer comprises a microstructure of nodes interconnected by fibrils, such as described in U.S. Pat. No. 3,953,566 to Gore. The fibrils radially extend from the nodes in a plurality of directions, and the membrane has a generally homogeneous structure. Membranes having this microstructure may typically exhibit a ratio of matrix tensile strength in two orthogonal directions of less than 2, and possibly less than 1.5.

In another embodiment, the expanded fluoropolymer membrane has a microstructure of substantially only fibrils, as is generally taught by U.S. Pat. No. 7,306,729, to Bacino. The expanded fluoropolymer membrane having substantially only fibrils, can possess a high surface area, such as greater than 20 m²/g, or greater than 25 m²/g, and in some embodiments can provide a highly balanced strength material having a product of matrix tensile strengths in two orthogonal directions of at least 1.5×10⁵ MPa², and/or a ratio of matrix tensile strengths in two orthogonal directions of less than 4, and possibly less than 1.5.

The expanded fluoropolymer membrane can be tailored to have any suitable thickness and mass to achieve the desired leaflet performance. By way of example, but not limited thereto, the leaflet 140 comprises an expanded fluoropolymer membrane having a thickness of about 0.1 μm. The expanded fluoropolymer membrane can possess a mass per area of about 1.15 g/m². Membranes according to an embodiment of the invention can have matrix tensile strengths of about 411 MPa in the longitudinal direction and 315 MPa in the transverse direction.

Additional materials may be incorporated into the pores or within the material of the membranes or in between layers of membranes to enhance desired properties of the leaflet. Composite materials described herein can be tailored to have any suitable thickness and mass to achieve the desired leaflet performance. Composite materials according to embodiments can include fluoropolymer membranes and have a thickness of about 1.9 μm and a mass per area of about 4.1 g/m².

The expanded fluoropolymer membrane combined with elastomer to form a composite material provides the elements of the present disclosure with the performance attributes required for use in high-cycle flexural implant applications, such as heart valve leaflets, in various ways. For example, the addition of the elastomer can improve the fatigue performance of the leaflet by eliminating or reducing the stiffening observed with ePTFE-only materials. In addition, it may reduce the likelihood that the material will undergo permanent set deformation, such as wrinkling or creasing, that could result in compromised performance. In one embodiment, the elastomer occupies substantially all of the pore volume or space within the porous structure of the expanded fluoropolymer membrane. In another embodiment the elastomer is present in substantially all of the pores of the at least one fluoropolymer layer. Having elastomer filling the pore volume or present in substantially all of the pores reduces the space in which foreign materials can be undesirably incorporated into the composite. An example of such foreign material is calcium that may be drawn into the membrane from contact with the blood. If calcium becomes incorporated into the composite material, as used in a heart valve leaflet, for example, mechanical damage can occur during cycling open and closed, thus leading to the formation of holes in the leaflet and degradation in hemodynamics.

In an embodiment, the elastomer that is combined with the ePTFE is a thermoplastic copolymer of tetrafluoroethylene (TFE) and perfluoromethyl vinyl ether (PMVE), such as described in U.S. Pat. No. 7,462,675 to Chang et al. As discussed above, the elastomer is combined with the expanded fluoropolymer membrane such that the elastomer occupies substantially all of the void space or pores within the expanded fluoropolymer membrane to form a composite material. This filling of the pores of the expanded fluoropolymer membrane with elastomer can be performed by a variety of methods. In one embodiment, a method of filling the pores of the expanded fluoropolymer membrane includes the steps of dissolving the elastomer in a solvent suitable to create a solution with a viscosity and surface tension that is appropriate to partially or fully flow into the pores of the expanded fluoropolymer membrane and allow the solvent to evaporate, leaving the filler behind.

In one embodiment, the composite material comprises three layers: two outer layers of ePTFE and an inner layer of a fluoroelastomer disposed therebetween. Additional fluoroelastomers can be suitable and are described in U.S. Publication No. 2004/0024448 to Chang et al.

In another embodiment, a method of filling the pores of the expanded fluoropolymer membrane includes the steps of delivering the filler via a dispersion to partially or fully fill the pores of the expanded fluoropolymer membrane.

In another embodiment, a method of filling the pores of the expanded fluoropolymer membrane includes the steps of bringing the porous expanded fluoropolymer membrane into contact with a sheet of the elastomer under conditions of heat and/or pressure that allow elastomer to flow into the pores of the expanded fluoropolymer membrane.

In another embodiment, a method of filling the pores of the expanded fluoropolymer membrane includes the steps of polymerizing the elastomer within the pores of the expanded fluoropolymer membrane by first filling the pores with a prepolymer of the elastomer and then at least partially curing the elastomer.

After reaching a minimum percent by weight of elastomer, the leaflets constructed from fluoropolymer materials or ePTFE generally performed better with increasing percentages of elastomer resulting in significantly increased cycle lives. In one embodiment, the elastomer combined with the ePTFE is a thermoplastic copolymer of tetrafluoroethylene and perfluoromethyl vinyl ether, such as described in U.S. Pat. No. 7,462,675 to Chang et al., and other references that would be known to those of skill in the art. Other biocompatible polymers which can be suitable for use in leaflet 140 include but are not limited to the groups of urethanes, silicones(organopolysiloxanes), copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of each of the foregoing.

### Leaflet

Each of the U-shaped portions 132 of the frame 130 is provided with the film 160 which can be coupled to the frame outside surface 133a and/or the frame inside surface 133b of the frame 130; wherein the film 160 defines a leaflet 140. Each leaflet 140 defines a leaflet free edge 142 that is not coupled to the frame 130.

The shape of the leaflets 140 are defined at least in part by the shape of the frame 130 and the leaflet free edge 142. The shape of the leaflets 140 can also be defined, at least in part, by processes used to manufacture the valve 100, such as, but not limited to a molding and trimming processes to impart a predetermined shape to the leaflet 140.

### Leaflet Dynamics

Fluid flow is permitted through the valve orifice 102 when the leaflets 140 are in an open position as shown in FIG. 1B. The leaflets 140 generally flex about the base 134 of the U-shaped portion 132 as the leaflets 140 open and close, as shown in FIG. 3. In an embodiment, when the valve 100 is closed, generally about half of each leaflet free edge 142 abuts an adjacent half of a leaflet free edge 142 of an adjacent leaflet 140, as shown in FIG. 2. The three leaflets 140 of the embodiment of FIGS. 1A and 2 meet at a triple point 148. The valve orifice 102 is occluded when the leaflets 140 are in the closed position stopping fluid flow.

The leaflet 140 can be configured to actuate at a pressure differential in the blood caused, for example, by the contraction of a ventricle or atrium of the heart, such pressure differential typically resulting from a fluid pressure building up on one side of the valve 100 when closed. As the pressure on an inflow side of the valve 100 rises above the pressure on the outflow side of the valve 100, the leaflet 140 opens and blood flows therethrough. As blood flows through the valve 100 into a neighboring chamber or blood vessel, the pressure equalizes. As the pressure on the outflow side of the valve 100 rises above the blood pressure on the inflow side of the valve 100, the leaflet 140 returns to the closed position generally preventing the retrograde flow of blood through the inflow side of the valve 100.

It is understood that the frame 130 can comprise any number of U-shaped portions 132, and thus leaflets 140, suitable for a particular purpose. Frames 130 comprising one, two, three or more U-shaped portions 132 and corresponding leaflets 140 are appreciated.

### Imageable Elements

Embodiments of leaflets presented herein comprise one or more imageable elements that allow for visualization of the movement of the leaflets using imaging techniques, such as, but not limited to fluoroscopy, x-ray, ultrasound, and MRI. The imageable element is suitable such that an imaging technique may be used after the prosthetic heart valve is deployed in a patient to visualize the movement of the leaflet to determine that the leaflet is functioning properly. Additionally, the imaging technique may be used postoperatively to visualize the movement of the leaflet to determine that the prosthesis is continuing to function properly. Imaging techniques such as X-ray, ultrasound, and MRI, may be used as a non-invasive technique without the dangers of a surgical procedure.

The following embodiments are presented describing the imageable element as being coupled to and contained within layers of the leaflet material. The following embodiments are presented describing the imageable element as a radiopaque marker, by way of example. The embodiments are also applicable to other imageable elements, such as echogenic elements, that are contained within layers of the leaflet material.

### Radiopaque Markers

Embodiments of leaflets presented herein comprise one or more imageable elements 150 in the form of radiopaque markers as shown in FIGS. 1A, 1B and 2. The radiopaque markers are operable to provide visualization under x-ray imaging so as to visualize the movement of the leaflet 140 as it moves between the open and closed positions. By providing visualization under x-ray imaging, the movement of the leaflet 140 between a substantially closed to open position, or vice versa, is helpful to confirm proper movement of the leaflets 140. For example, the imageable elements 150 in the form of radiopaque markers in accordance with the present embodiments may facilitate the observation that one or more of the leaflets 140 is immobile requiring further intervention to provide proper valve function. Further, for example, the imageable elements 150 in the form of radiopaque markers in accordance with the present embodiments may facilitate the observation that one or more of the leaflets 140 is moving in am improper manner preventing coaptation of the leaflets requiring further intervention to provide proper valve function.

The imageable element 150 in the form of a radiopaque marker is any element that is within the leaflet 140 between layers of film 160 that provides visualization under x-ray imaging. The imageable element 150 in the form of a radiopaque marker is operable to provide minimal or no structural characteristics to the leaflet 140.

In an exemplary embodiment, an imageable element 150 in the form of a radiopaque marker may comprise any shape, any configuration, or any material operable to provide visualization under x-ray imaging. For example, an imageable element 150 in the form of a radiopaque marker may comprise at least one of a film, a foil, and a powder.

The radiopaque marker may comprise any material suitable for the particular purpose. For example, a high density metal such as platinum, gold, tungsten, barium, bismuth, and tantalum may be used in a sufficient quantity that the radiopaque marker is readily visible and distinguishable from adjacent structure under x-ray imaging. The high density metal may be in any suitable form, such as, but not limited to, foil, powder, and wire.

It is believed that despite the presence of additional minimal mass added to the leaflet 140 by the imageable element 150 in the form of a radiopaque marker, the leaflet 140 comprising the radiopaque marker remains responsive to changes in fluid pressure.

In an embodiment, the shape of the imageable element 150 in the form of a radiopaque marker comprises an oval, as shown in FIG. 1A. Alternative configurations are appreciated, such as, but not limited to, a geometric shape, such as a circle, triangle, square, and rectangle, an undulating shape, an S-shape.

In another embodiment, the radiopaque marker may be a distribution of a powder that is coupled to and contained between layers of the film 160. In such embodiments, the distribution of powder may define a shape or be substantially distributed across the leaflet.

In another embodiment, the radiopaque marker may be a polymer resin coating that includes a radiopaque filler material such as gold, barium sulfate, bismuth and tungsten powder. The coating is applied to and contained between layers of the film 160. In such embodiments, the distribution of the radiopaque filler material may define a shape or be substantially distributed across the leaflet.

Irrespective of the shape of the marker employed, an important attribute is the aspect ratio of the dimensions of the marker in the leaflet plane as compared to the thickness of the marker. Preferably the thickness of the marker is less that about 10% of the length of the marker in either axial or circumferential directions. In other embodiments the aspect ratio is less than 0.05. Thinner markers provide lower mass and allow superior lamination of layers as compared to thicker markers. This aspect ratio requirement thus excludes clips placed on the edge of the leaflet.

Similarly, a leaflet 140 need not be limited to one imageable element 150 in the form of a radiopaque marker per leaflet 140. Rather any number of radiopaque markers may be present, and the present embodiments contemplate any radiopaque marker comprising any material of any combination that provides visualization under x-ray imaging.

In an exemplary embodiment, the radiopaque marker may comprise a plurality of materials and be located in a plurality of locations within the leaflet 140, and thereby provide visualization under x-ray imaging suitable for a particular purpose. By way of example, a radiopaque marker of one type of material may be present adjacent the base of the leaflet 140 while another radiopaque marker of another type of material may be present adjacent the leaflet free edge 142 so as to provide visual information relating to the base and the free edge, respectively

### Location of Markers

Referring again to FIG. 1A, a midline Lm is provided that extends from one post 131 to an adjacent post 131 that substantially divides the leaflet 140 through a leaflet central portion 165 into a leaflet base portion 161 adjacent the leaflet base 135 and a leaflet free edge portion 163 adjacent the leaflet free edge 142. A radial line Lr is defined that extends from the triple point 148 in a radial direction.

In accordance with the embodiment of FIG. 1A, an imageable element 150 in the form of a radiopaque marker is located within the leaflet free edge portion 163, along the radial line Lr and adjacent the triple point 148. In such a location, proper coaptation of the leaflets 140 may be inferred by observing using x-ray techniques that the radiopaque markers meet at or are adjacent to the triple point 148 when the valve 100 is in the closed position. The valve orifice 102 is occluded when the leaflets 140 are in the closed position and forming the triple point 148 which stops fluid flow. Improper coaptation of the leaflets 140, or malfunctioning leaflet movement caused by such things as, but not limited to, thrombus, calcification, and material failure, may be inferred by observing using x-ray techniques that the radiopaque markers are not moving or meeting as expected.

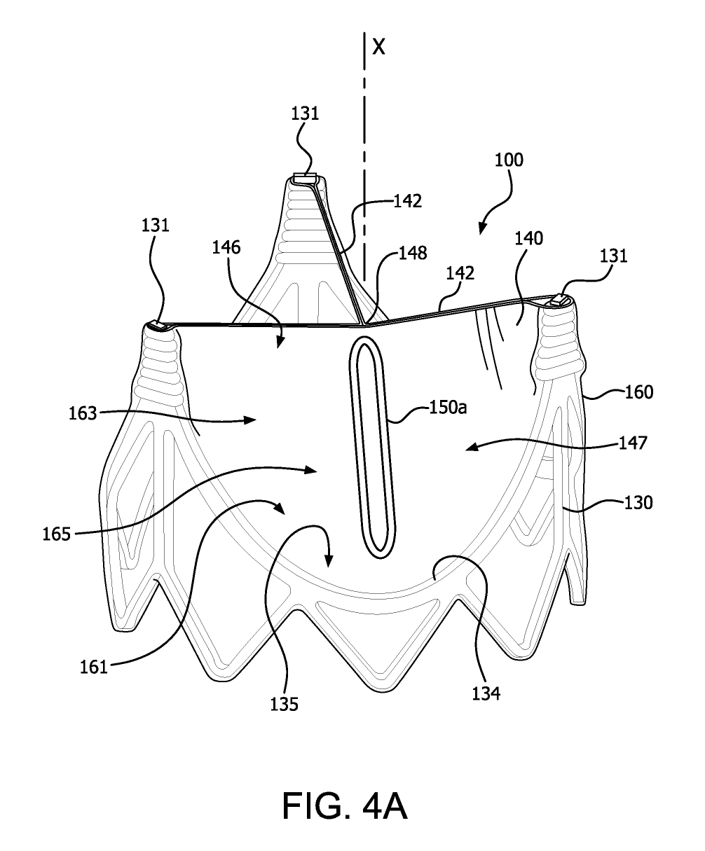

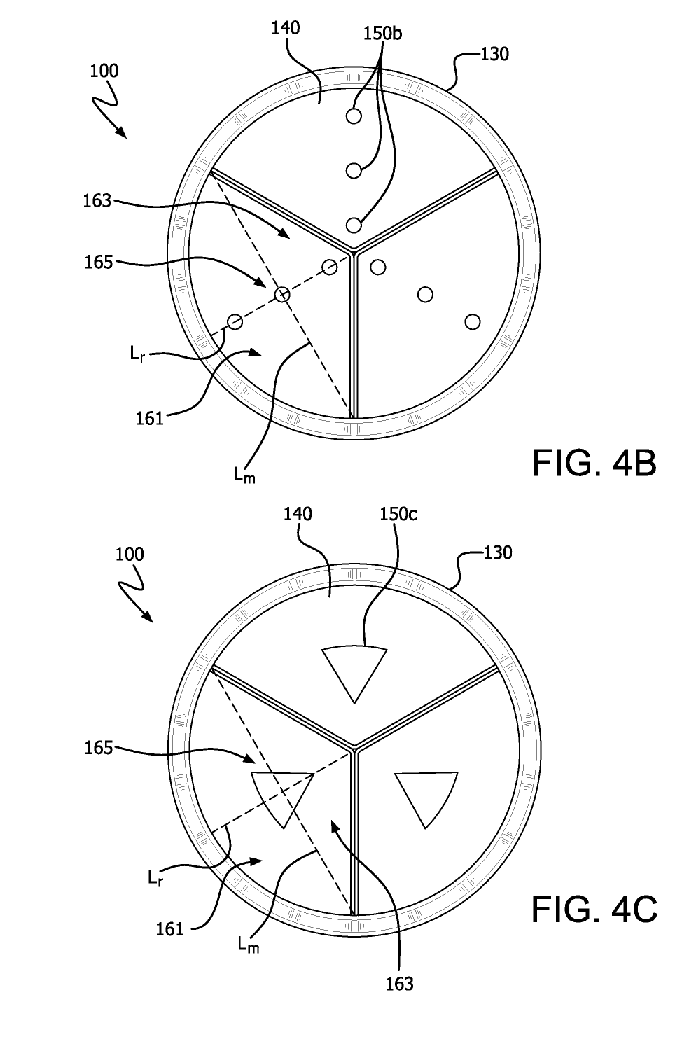

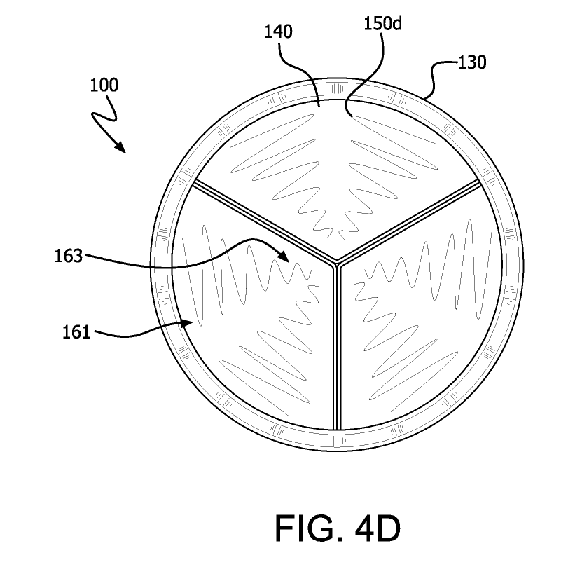

FIG. 4A is a perspective view of a valve 100 wherein the imageable element 150a is an elongated member that is aligned along the radial line Lr and extends within the leaflet base portion 161 and the leaflet free edge portion 163 through the leaflet central portion 165, in accordance with the embodiment.

FIG. 4B is an axial view of a valve 100 wherein the leaflet 140 includes three imageable elements 150b that are disk members that are aligned along the radial line Lr and extend within the leaflet base portion 161, the leaflet free edge portion 163, and the leaflet central portion 165 respectively, in accordance with the embodiment.

FIG. 4C is an axial view of a valve 100 wherein the imageable element 150c is a triangular member that is aligned along the radial line Lr and extends within the leaflet central portion 165, in accordance with the embodiment.

FIG. 4D is an axial view of a valve 100 wherein the imageable element 150d is a powder that is substantially uniformly distributed within the leaflet base portion 161, the leaflet free edge portion 163, and the leaflet central portion 165, in accordance with the embodiment.

### Method of Making

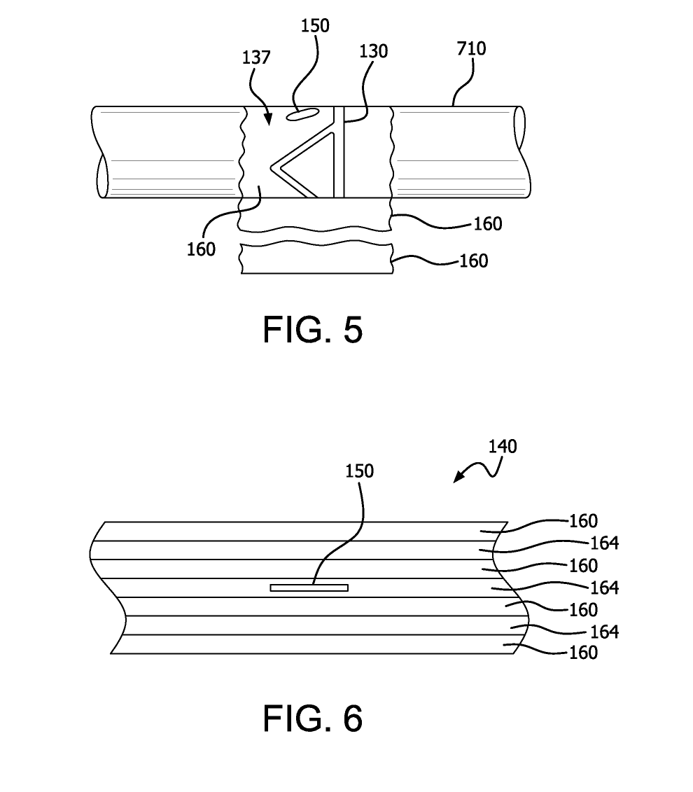

Embodiments described herein, by way of examples below, also pertain to a method of making the valve 100 embodiments as described herein comprising imageable elements 150 in the form of radiopaque markers. In order to make the various embodiments, a cylindrical mandrel can be used. With reference to FIG. 5, the mandrel 710 comprises a structural form operable to receive the frame 130 thereon, in accordance with the embodiment. The film 160 is wound onto the mandrel, under and/or over the frame 130 to define a laminate. An imageable element 150 in the form of a radiopaque marker, for example, may be coupled to the film 160 during a winding process so as to encapsulate the imageable element 150 between the layers of film 160 of the leaflet 140, as shown in cross-sectional view in FIG. 6, in accordance with the embodiment. Referring to FIG. 6, the leaflet 140 comprises multiple layers of film 160 coupled together with elastomeric material 164 therebetween.

### EXAMPLE

A heart valve having polymeric leaflets containing radiopaque markers was formed from a composite material having an expanded fluoropolymer membrane and an elastomeric material and joined to a metallic frame, and was constructed according to the following process:

A valve frame was laser machined from a length of MP35N cobalt chromium tube hard tempered with an outside diameter of 26.0 mm and a wall thickness of 0.6 mm in the shape shown in FIG. 3. The frame was electro-polished resulting in 0.0127 mm material removal from each surface and leaving the edges rounded. The frame was exposed to a surface roughening step to improve adherence of leaflets to the frame, without degrading fatigue durability performance. The frame was cleaned by submersion in an ultrasonic bath of acetone for approximately five minutes. Plasma treatment of the entire metal frame surface was performed as commonly known in the arts for cleaning. This treatment also served to improve the wetting of the fluorinated ethylene propylene (FEP) adhesive.

FEP powder (Daikin America, Orangeburg N.Y.) was applied to the frame by first stirring the powder into an airborne “cloud” in a standard kitchen type blender and suspending the frame in the cloud until a uniform layer of powder adhered to the entire surface of the frame. The frame was then subjected to a thermal treatment by placing it in a forced air oven set to 320° C. for approximately three minutes. This caused the powder to melt and adhere as a thin coating over the entire frame. The frame was removed from the oven and left to cool to room temperature.

A strain relief and sewing ring were attached to the frame in the following manner: a 23 mm diameter cylindrical mandrel was wrapped with a single layer of Kapton® (DuPont) polyimide film and held in place by an adhesive strip of Kapton® tape over the length of the overlapping seam. One wrap of a two layer laminate consisting of an ePTFE membrane laminated to a 25.4 μm thick layer of fluoroelastomer was wrapped with the high strength direction along the axis of the Kapton®—covered mandrel with no overlap at the seam. The frame was aligned coaxially over the wrapped mandrel. An additional one wrap of the two layer laminate was wrapped onto the mandrel encapsulating the entire frame with the seam oriented 180° from the seam of the single inner wrap. The four layer laminate was end cut 135 mm from the base of the frame encapsulated within. The four layer laminate was hand rolled axially in the direction of the base of the frame until the 135 mm length of material constituted approximately a 3 mm outer diameter ring adjacent to the base of the frame. The four layer laminate was end cut approximately 20 mm from the top of the frame and the assembly was compression wrapped helically with two sacrificial layers of ePTFE membrane imbibed with a polyimide, four layers of unsintered ePTFE membrane, and approximately one hundred wraps of an ePTFE fiber. The entire assembly was subjected to a thermal treatment by placing it in a forced air oven set to 280° C. for five minutes and returned to room temperature by immediate water quench upon removal from the oven. The sacrificial layers were removed and the four layer laminate at the top end of the frame trimmed to allow a 2 mm length to extend beyond the perimeter of the top of the frame. The mandrel and Kapton were then removed from the interior of the frame forming a strain relief and sewing ring with the frame laminated within.

A single female mold defining the shape of the tri-leaflet was made. Three identical male molds that match the shape and contour of the female mold are held together with a mechanism that enables radial pivoting of the male molds with respect to each other at their base while maintaining both axial and rotational spacing. The female and male molds are wrapped with a single layer of un-sintered ePTFE membrane to act as a cushioning layer and then a single layer of substantially nonporous ePTFE membrane with FEP on one side is used to adhere the membranes together and onto the mandrels with a soldering iron. The sacrificial layers ensure that all the mating surfaces between the male and female molds have a cushioning layer when compressed together; an additional function is as a release layer to prevent the leaflet material from adhering to the molds. The male and female molds are initially combined to create a single cylindrical structure to facilitate leaflet construction and attachment to the frame with strain relief and sewing ring component via a tape wrapping process.

A leaflet material was then prepared. A membrane of ePTFE was manufactured according to the general teachings described in U.S. Pat. No. 7,306,729. The ePTFE membrane had a mass per area of 1.0 g/m2 a matrix tensile strength of 447 MPa in the longitudinal direction and 421 MPa in the transverse direction.

The above membrane was imbibed with a copolymer fluoroelastomer. The copolymer consists essentially of between about 65 and 70 weight percent perfluoromethyl vinyl ether and complementally about 35 and 30 weight percent tetrafluoroethylene. Additional fluoroelastomers may be suitable and are described in U.S. Publication No. 2004/0024448. The fluoroelastomer was dissolved in Novec HFE7500 (3M, St Paul, Minn.) in a 2.5% concentration. The solution was coated using a mayer bar onto the ePTFE membrane (while being supported by a polypropylene release film) and dried in a convection oven set to 145° C. for 30 seconds. After 2 coating steps, the final ePTFE/fluoroelastomer or composite had a mass per area of 6.92 g/m2, 14.4% fluoropolymer by weight, and thickness of 3.22 μm.

Five layers of the composite material was wrapped around the combined molds with the membrane oriented such that the matrix tensile strength of 447 MPa is oriented axially and the elastomer rich side of the composite facing away from the molds.

The subassembly containing the frame with strain relief was aligned both axially and rotationally to match the features of the female mold over the three inner wraps. Ten additional layers of the composite material were wrapped around the combined molds with the membrane oriented such that the matrix tensile strength of 410.9 MPa was oriented axially and the elastomer rich side of the composite facing toward the molds.

Three radiopaque markers were incorporated into the laminated leaflet construction between the 3rd and 4th layers of the composite material so that they were contained entirely within the layers of each of the three leaflets. These were constructed of gold foil that was 0.005 mm thick, in the shape of an oval having dimensions of 1 mm by 0.13 mm with an aspect ratio of 0.125. These elements were located in the leaflet region as shown in FIG. 2.

The male molds were then slid out from underneath the 15-layer composite laminate tube. Each of the male molds was expanded with respect to each other about the pivot at their base. The male mold assembly was coaxially aligned to the female mold facilitating the male molds to compress the cantilevered 15-layer composite laminate tube onto the female tri-leaflet mold surface. Both radial and axial compression were applied by placing a hose clamp over the male molds while simultaneously applying axial load with the translational end of the lathe apparatus.

The assembly consisting of male and female molds, composite laminate, strain relief, frame, and sewing ring was compression wrapped helically with two sacrificial layers of compliant ePTFE membrane imbibed with a polyimide, four layers of un-sintered ePTFE membrane, and approximately one hundred wraps of an ePTFE fiber. The entire assembly was removed from the lathe and placed in a c-clamp fixture to maintain axial compression while subjected to a thermal treatment by placing it in a forced air oven set to 280° C. for 30 minutes. The assembly was removed from the oven and brought back to room temperature via immediate water quench. The sacrificial layers, male, and female molds were removed leaving a fully adhered valve in a closed three dimensional form.

The excess leaflet material was trimmed with scissors from the top of the frame posts to the common triple point of each leaflet to create three commissures or coapting surface regions as depicted in FIG. 2.

The final leaflet was comprised of 14.4% fluoropolymer by weight with a thickness of 58 μm. Each leaflet had 15 layers of the composite and a ratio of thickness/number of layers of 3.87 μm.

The resulting valve assembly includes leaflets formed from a composite material with more than one fluoropolymer layer having a plurality of pores and an elastomer present in substantially all of the pores of the more than one fluoropolymer layer. Each leaflet is capable of being cycled between a closed position, shown illustratively in FIG. 1A, in which blood is prevented from flowing through the valve assembly, and an open position, shown illustratively in FIG. 1B, in which blood is allowed to flow through the valve assembly. Thus, the leaflets of the valve assembly cycle between the closed and open positions generally to regulate blood flow direction in a human patient.

The performance of the radiopaque markers in the valve leaflets in each valve assembly was characterized using fluoroscopy after the valve was implanted in an animal model.

The performance of the valve leaflets in each valve assembly was characterized on a real-time pulse duplicator that measured typical anatomical pressures and flows across the valve. No significant dynamic performance difference was noticed between valves having leaflets with and without the radiopaque marker.

### Echogenic Elements

As previously discussed, the embodiments discussed above are also applicable to other imageable elements, such as echogenic elements that comprise echogenic material, that are contained between at least two layers of film that makes up the leaflet material. The echogenic elements may be any element that is ultrasonically reflective, that is, operable to be visualized using ultrasound imaging techniques. When visualized using ultrasound imaging techniques, the movement of the echogenic element is directly related to the movement of the leaflet to which it is coupled, and therefore the movement of the leaflet may be determined.

The echogenic element may be substantially the same as the imageable elements 150 in the form of radiopaque markers as discussed above and may comprise any material suitable for the particular purpose. For example, a high density metal such as platinum, gold, tungsten, barium, bismuth, and tantalum may be used in a sufficient quantity that the echogenic element is readily visible and distinguishable from adjacent structure under ultrasound imaging techniques.

The foregoing disclosure is merely illustrative of the present invention and is not intended to be construed as limiting the invention. Although one or more embodiments of the present invention have been described, persons skilled in the art will readily appreciate that numerous modifications could be made without departing from the spirit and scope of the present invention. As such, it should be understood that all such modifications are intended to be included within the scope of the present invention.

What is claimed is:

1. A prosthetic valve, comprising:
a leaflet comprising a least two layers of film; and
at least one imageable element coupled to and contained between at least two of the at least two layers of film.

2. The prosthetic valve of claim 1, wherein the at least one imageable element is selected from a list consisting of a radiopaque marker and an echogenic element.

3. The prosthetic valve of claim 1, wherein the at least one imageable element comprises radiopaque material.

4. The prosthetic valve of claim 1, wherein the at least one imageable element comprises an echogenic material.

5. The prosthetic valve of claim 1, wherein the at least one imageable element comprises a metal foil.

6. The prosthetic valve of claim 1, wherein the at least one imageable element comprises a powder.

7. The prosthetic valve of claim 1, wherein the at least one imageable element defines a size and is of sufficient quantity that the imageable element is readily visible and distinguishable from adjacent structure using imaging techniques.

8. The prosthetic valve of claim 1, wherein the at least one imageable element defines a size and is of sufficient quantity that the imageable element is readily visible and distinguishable from adjacent structure using x-ray imaging techniques.

9. The prosthetic valve of claim 1, wherein the at least one imageable element defines a size and is of sufficient quantity that the imageable element is readily visible and distinguishable from adjacent structure using ultrasound imaging techniques.

10. The prosthetic valve of claim 1, wherein the at least one imageable element comprises a thickness of less than 0.005 mm operable to not interfere with a movement of the leaflet.

11. The prosthetic valve of claim 1, wherein the at least one imageable element is not as stiff as one layer of the film.

12. The prosthetic valve of claim 1, wherein the at least one imageable element comprises a metallic material.

13. The prosthetic valve of claim 12, wherein the metallic material is selected from a list consisting of platinum, gold, tungsten, barium, bismuth, silver and tantalum.

14. The prosthetic valve of claim 1, wherein the at least one imageable element defines a shape selected from a list consisting of a circle, oval, triangle, and square.

15. The prosthetic valve of claim 1, wherein the leaflet defines a leaflet base portion and a leaflet free edge portion opposite from the leaflet base portion, one of the imageable elements being located within the leaflet base portion and another one of the imageable elements being located within the leaflet free edge portion.

16. The prosthetic valve of claim 1, wherein the leaflet comprises a composite material having at least two fluoropolymer layers, each fluoropolymer layer having a plurality of pores and an elastomer present in substantially all of the pores, wherein the imaging element is coupled to and contained between at least two of the at least two fluoropolymer layers.

17. The prosthetic valve of claim 16, wherein at least one fluoropolymer layer comprises a plurality of pores.

18. The prosthetic valve of claim 17, wherein substantially all of the pores contain an elastomer.

19. The prosthetic valve of claim 18, wherein the elastomer comprises a fluoroelastomer.

20. The prosthetic valve of claim 19, wherein the elastomer comprises a TFE/PMVE copolymer.

21. The prosthetic valve of claim 16, wherein the fluoropolymer comprises PTFE.

22. The prosthetic valve of claim 21, wherein the PTFE is ePTFE.
